(19) United States
(12) Patent Application Publication
(10) Pub. No.: US 2023/0147129 A1
(43) Pub. Date: May 11, 2023

(54) COMPOUNDS FOR USE IN THE TREATMENT OF SYNUCLEINOPATHIES

(71) Applicants: SOM INNOVATION BIOTECH, S.A., Barcelona (ES); UNIVERSITAT AUTÒNOMA DE BARCELONA, Cerdanyola de Vallès, Barcelona (ES)

(72) Inventors: Raúl INSA BORONAT, Barcelona (ES); Núria REIG BOLAÑO, Barcelona (ES); Luca SIGNORILE, Barcelona (ES); Oscar HUERTAS GAMBÍN, Barcelona (ES); Salvador VENTURA ZAMORA, Bellaterra, Cerdanyola del Vallès (ES); Samuel PEÑA DÍAZ, Bellaterra, Cerdanyola del Vallès (ES)

(21) Appl. No.: 17/907,014

(22) PCT Filed: Mar. 26, 2021
(86) PCT No.: PCT/EP2021/057907

§ 371 Date: Sep. 22, 2022

(30) Foreign Application Priority Data
Mar. 27, 2020 (EP) 20382239.0

(51) Int. Cl. A61K 31/635; A61K 31/36; A61K 31/4168; A61K 31/4155; A61K 31/522; A61K 31/357; A61K 31/4184; A61K 31/513; A61K 31/137; A61K 31/175; A61K 31/165; A61P 25/16

(52) U.S. Cl. CPC A61K 31/635; A61K 31/36; A61K 31/4168; A61K 31/4155; A61K 31/522; A61K 31/357; A61K 31/4184; A61K 31/513; A61K 31/137; A61K 31/175; A61K 31/165; A61P 25/16

(57) ABSTRACT

The present invention relates to a compound selected from the group consisting of of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide and 2-(1,4-dioxaspiro[4.5]decan-2-ylmethyl)guanidin, or a pharmaceutically acceptable salt thereof and combinations comprising at least one of said compounds, for use in the treatment and/or prevention of synucleinopathies.

### FIELD OF THE INVENTION

The present invention relates to compounds and combinations thereof useful in the treatment and/or prevention of synucleinopathies such as Parkinson's disease.

### BACKGROUND OF THE INVENTION

α-Synuclein is a 140 aminoacids-long protein, being especially abundant in the brain, mainly at the presynaptic terminals of neurons, with minor amounts found in other tissues such as muscle and heart. Although its physiological role is currently not completely understood, it has been suggested to help regulate the release of dopamine, and to play a role in restricting the mobility of synaptic vesicles. It is an intrinsically disordered protein, adopting an α-helix conformation when bound to membranes.

The abnormal accumulation of aggregates of alpha-synuclein protein in neurons, nerve fibers or glial cells is the underlying cause of synucleinopathies, which are often classified in three main types: Parkinson's disease (PD), dementia with Lewy bodies (DLB), and multiple system atrophy (MSA) although there are other rare disorders, such as various neuroaxonal dystrophies that also involve α-synuclein aggregates.

The value of α-synuclein as a therapeutic target is validated by the number of clinical trials currently ongoing to assess the efficacy of different technologies targeting this protein, such as antibodies (prasinezumab from Roche, and BIIB054 from Biogen), vaccines (PD01A and PD03A from Affiris) and small-molecule inhibitors (NPT088 from Proclara Biosciences).

Parkinson's disease (PD), the most prevalent synucleinopathy, is the second most common neurodegenerative disorder behind Alzheimer's disease. It is a long-term and progressive neurological disorder, mainly affecting the motor system, although at later stages non-motor symptoms become increasingly common.

The pathophysiology of PD is characterized by extensive cell death in the basal ganglia of the brain, hence resulting in a dopamine deficiency in this region, and the presence of Lewy bodies and Lewy neurites in many of the remaining neurons. Indeed, the presence of these pathological structures is one of the gold standards for the diagnosis of Parkinson's disease at post-mortem pathological examination.

Currently, there is no cure for Parkinson's disease and its cause is still the subject of intense research, although it appears to involve both genetic and environmental factors. Clinically, it manifests with the characteristic “parkinsonism” motor symptomatology: tremors, rigidity, postural instability, bradykinesia (reduced movements, and slow initiation of voluntary movements). Non-motor symptoms, which in some cases can precede motor dysfunction by more than a decade, usually include dementia (at later stages), depression, anxiety, as well as sleep and emotional disturbances.

Therapeutic approaches focus on the improvement of the symptomatology, usually the motor-system manifestations due to reduced dopamine levels in the central nervous system (CNS), thus attempting to supplement and/or maintain dopamine levels, through the intake of dopamine precursors (levodopa), stimulants of dopamine receptors, and through the inhibition of dopamine-degrading enzymes, such as monoamine oxidase B (MAO-B) and catechol-O-methyltransferase.

Despite the above, currently there is a small choice of pharmacological agents for the treatment of synucleinopathies such as Parkinson's disease (PD), and these focus on symptomatic improvement, and have considerable side effects, hence there is a need for new pharmacological disease-modifying agents useful for the treatment of said synucleinopathies.

### SUMMARY OF THE INVENTION

The inventors have surprisingly found new pharmacological strategies for the treatment of synucleinopathies such as Parkinson's disease, by identifying compounds that have shown a positive effect on the aggregation of α-synuclein protein, i.e. preventing and/or inhibiting its aggregation.

Thus, in a first aspect, the present invention relates to a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof, for use in the treatment and/or prevention of synucleinopathies with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a second aspect, the present invention relates to the use of a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof, in the manufacture of a medicament for the treatment and/or prevention of synucleinopathies with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a third aspect, the present invention relates to a method of treating and/or preventing synucleinopathies in a subject, comprising administering to said subject a therapeutically effective amount of a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a fourth aspect, the present invention relates to a combination comprising one or more compounds selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof, for use in the treatment and/or prevention of synucleinopathies, with the proviso that, when the compound is cinacalcet (i.e. N-[(1R)-1-(1-naphthyl)ethyl]-3-[3-(trifluoromethyl)phenyl]propan-1-amine), the treatment and/or prevention are not carried out in subjects having an abnormal calcium metabolism such as hypercalcemia or hyperparathyroidism and wherein at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof.

### DESCRIPTION OF THE INVENTION

In the first aspect, the present invention relates to a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide or a pharmaceutically acceptable salt thereof, for use in the treatment and/or prevention of synucleinopathies with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a second aspect, the present invention also relates to the use of a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide or a pharmaceutically acceptable salt thereof, in the manufacture of a medicament for the treatment and/or prevention of synucleinopathies with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a third aspect, the present invention also relates to a method of treating and/or preventing synucleinopathies in a subject, comprising administering to said subject a therapeutically effective amount of a compound selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism.

In a particular embodiment of the first, second or third aspects mentioned above, the compounds are used as sole active ingredients.

In a particular embodiment of the first, second or third aspects mentioned above, the compound is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide and 2-(1,4-dioxaspiro[4.5]decan-2-ylmethyl)guanidine, or a pharmaceutically acceptable salt thereof.

In another embodiment of the first, second or third aspects mentioned above, the compound is selected from the group consisting of 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof.

In a particular embodiment of the first, second and third aspects mentioned above, the synucleinopathy to be treated and/or prevented is selected from the group consisting of Parkinson's disease (PD), Multiple System Atrophy (MSA) and Dementia with Lewy Bodies. Preferably, the synucleinopathy to be treated and/or prevented is Parkinson's disease (PD).

The terms “treating” and “treatment”, as used herein, means reversing, alleviating, inhibiting the progress of, the disease or condition to which such term applies, or one or more symptoms of such disease or condition, such preventing or inhibiting the aggregation of α-synuclein with respect to pretreatment levels.

The terms “preventing” and “prevention”, as used herein, means avoiding or inhibiting the onset of one or more symptoms of the disease or condition to which such term applies.

The terms “synucleinopathy”, and “synucleinopathies” as used herein, refer to neurodegenerative diseases characterized by the abnormal accumulation of aggregates of alpha-synuclein protein in neurons, nerve fibers or glial cells. Examples of said diseases are Parkinson's disease (PD), dementia with Lewy bodies (DLB) and multiple system atrophy (MSA), preferably Parkinson's disease (PD).

1,2-bis[(4-chlorophenyl)methylideneamino]guanidine has the chemical structure depicted below.

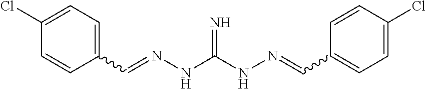 [SMILES: N=C(NN=Cc1ccc(Cl)cc1)NN=Cc1ccc(Cl)cc1]

This compound has received the INN name of robenidine, and was developed as an antiparasitic agent, exhibiting antiprotozoal activity against Coccidia parasites. This compound is commercially available or may be synthesized using a suitable preparation method, such as that disclosed in U.S. Pat. No. 6,680,409 B2. Although both isomers and/or their mixtures are within the scope of the present invention, preferably, the (E) isomer, 1,2-bis[(E)-(4-chlorophenyl)methylideneamino]guanidine, is used.

N-[1-(1-naphthyl)ethyl]-3-[3-(trifluoromethyl)phenyl]propan-1-amine has the chemical structure depicted below.

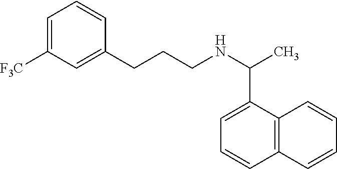 [SMILES: CC(NCCCc1cccc(C(F)(F)F)c1)c1cccc2ccccc12]

The (R) enantiomer, i.e. N-[(1R)-1-(1-naphthyl)ethyl]-3-[3-(trifluoromethyl)phenyl]propan-1-amine, having the INN name Cinacalcet is a calcimimetic, currently marketed by Amgen in Europe as Mimpara® and in the US as Sensipar®. This compound is commercially available, or may be synthesized using a suitable preparation method, such as that disclosed in U.S. Pat. No. 6,211,244 B1. Although both enantiomers and/or their mixtures are within the scope of the present invention, preferably, the (R) isomer is used.

N-[1-amino-3-(3-fluorophenyl)propan-2-yl]-5-chloro-4-(4-chloro-1-methyl-H-pyrazol-5-yl)thiophene-2-carboxamide has the chemical structure depicted below.

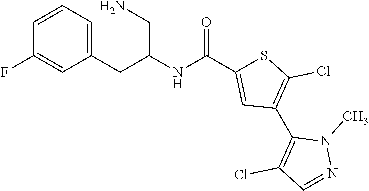 [SMILES: Cn1ncc(Cl)c1-c1cc(C(=O)NC(CN)Cc2cccc(F)c2)sc1Cl]

The (S) isomer, i.e. N-[(2S)-1-amino-3-(3-fluorophenyl)propan-2-yl]-5-chloro-4-(4-chloro-1-methyl-1H-pyrazol-5-yl)thiophene-2-carboxamide, known as afuresertib, is a compound initially developed by GSK as an anti-cancer agent, being a protein kinase B (Akt) inhibitor. Said compound is commercially available or may be synthesized using a suitable preparation method, such as the one disclosed in EP 2 117 523 B1. Although both enantiomers and/or their mixtures are within the scope of the present invention, preferably, the (S) isomer is used.

[2-(2,6-dichloroanilino)-4,5-dihydroimidazol-1-yl]-phenylmethanone, also known by the INN name of benclonidine, has the chemical structure depicted below.

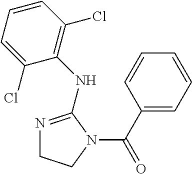 [SMILES: O=C(c1ccccc1)N1CCN=C1Nc1c(Cl)cccc1Cl]

This compound has been developed as an anti-hypertensive drug, additionally displaying moderate sedative effects. This compound is commercially available or may be synthesized using a suitable preparation method, such as that disclosed in GB 1,506,914.

Methyl N-(6-propoxy-1H-benzimidazol-2-yl) carbamate, having the INN name oxibendazole, has the chemical structure depicted below.

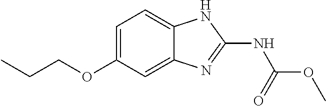 [SMILES: CCCOc1ccc2[nH]c(NC(=O)OC)nc2c1]

This compound has been developed as an anthelmintic agent. This compound is commercially available or may be synthesized using a suitable preparation method, such as that disclosed in GB 1,123,317.

[4-(2-(diaminomethylidene)hydrazinyl)phenyl]iminothiourea, having the INN name ambazone, has the chemical structure shown below.

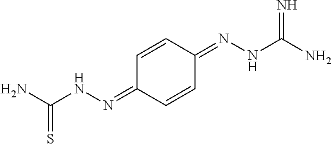 [SMILES: N=C(N)NN=C1C=CC(=NNC(N)=S)C=C1]

This compound has been developed by Bayer as an antibiotic (bacteriostatic action against Streptococcus), and has been additionally shown to have anti-tumor activity, against leukemia in mice. This compound is commercially available or may be synthesized using a suitable preparation method, such as that disclosed in DE 965723.

N-(diaminomethylidene)-2-(2,6-dichlorophenyl)acetamide, having the INN name guanfacine, has the chemical structure depicted below.

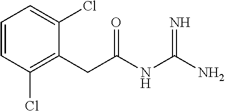 [SMILES: N=C(N)NC(=O)Cc1c(Cl)cccc1Cl]

This compound has been developed as an anti-hypertension drug, although recently it has been approved for the treatment of attention deficit disorder (ADHD). This compound is commercially available or may be synthesized using a suitable preparation method, such as that disclosed in GB 1,235,723.

2-amino-5-bromo-4-phenyl-1H-pyrimidin-6-one, having the INN name bropirimine, has the chemical structure depicted below.

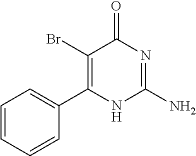 [SMILES: Nc1nc(=O)c(Br)c(-c2ccccc2)[nH]1]

This compound has been shown to have anti-cancer (bladder) and anti-viral activities, being an effective immunomodulator. This compound is commercially available or may be synthesized using a suitable preparation method, such as that disclosed in Skulnick, H., et al., J. Med. Chem. 1985, 28, 1864-1869.

2-(1,4-dioxaspiro[4.5]decan-2-ylmethyl)guanidine, having the INN name guanadrel, has the chemical structure depicted below.

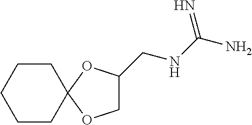 [SMILES: N=C(N)NCC1COC2(CCCCC2)O1]

This compound has been developed as an anti-hypertensive agent. This compound is commercially available or may be synthesized using a suitable preparation method, such as that disclosed in U.S. Pat. No. 3,547,951. Both enantiomers and/or their mixtures are within the scope of the present invention.

5-[2-[[3-(1,3-Benzodioxol-5-yl)-1-methylpropyl]amino]-1-hydroxyethyl]-2-hydroxybenzamide, having the INN name medroxalol, has the chemical structure depicted below.

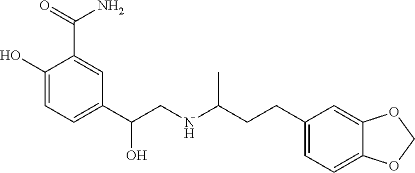 [SMILES: CC(CCc1ccc2c(c1)OCO2)NCC(O)c1ccc(O)c(C(N)=O)c1]

This compound has been developed as an anti-hypertensive agent. This compound is commercially available or may be synthesized using a suitable preparation method, such as that disclosed in U.S. Pat. No. 3,883,560. Both enantiomers and/or their mixtures are within the scope of the present invention.

2-amino-9-[4-(hydroxymethyl)cyclopent-2-en-1-yl]-1H-purin-6-one has the chemical structure depicted below.

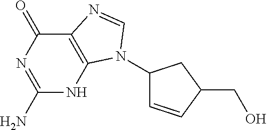 [SMILES: Nc1nc(=O)c2ncn(C3C=CC(CO)C3)c2[nH]1]

This compound is a guanosine analog with activity as reverse transcriptase inhibitor that was developed as an anti-viral. It is commercially available or may be synthesized using a suitable preparation method, such as that disclosed in DE 3901502. Although all stereoisomers and/or their mixtures are within the scope of the present invention, preferably, the (1R,4S) isomer, i.e. 2-amino-9-[(1R,4S)-4-(hydroxymethyl)cyclopent-2-en-1-yl]-1H-purin-6-one, commonly known as (−)-carbovir is used.

Alternatively, the prodrug of 2-amino-9-[4-(hydroxymethyl)cyclopent-2-en-1-yl]-1H-purin-6-one can be used, having the chemical name of 4-[2-amino-6-(cyclopropylamino)-9H-purin-9-yl]-2-cyclopentene-1-methanol and the chemical structure depicted below.

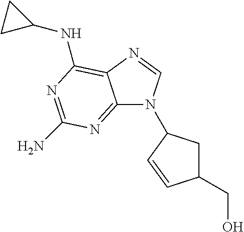 [SMILES: Nc1nc(NC2CC2)c2ncn(C3C=CC(CO)C3)c2n1]

Although all stereoisomers and/or their mixtures are within the scope of the present invention, preferably the (1S,4R) isomer, i.e. (1S,4R)-4-[2-amino-6-(cyclopropylamino)-9H-purin-9-yl]-2-cyclopentene-1-methanol, commonly known as abacavir or (−)-abacavir, is used. This compound is commercialized for the treatment of HIV as a prodrug of (−)-carbovir.

4-amino-N-(4-methylpyrimidin-2-yl)benzenesulfonamide, having the INN name sulfamerazine, has the chemical structure depicted below.

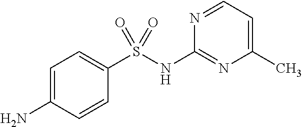 [SMILES: Cc1ccnc(NS(=O)(=O)c2ccc(N)cc2)n1]

This compound belongs to the sulfonamide antibiotics class of drugs. This compound is commercially available or may be synthesized using a suitable preparation method, such as that disclosed in U.S. Pat. No. 2,688,015.

The term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

Further, the term “pharmaceutically acceptable salt” refers to any salt, which, upon administration to the recipient is capable of providing (directly or indirectly) a compound as described herein. For instance, a pharmaceutically acceptable salt of compounds provided herein may be acid addition salts, base addition salts or metallic salts, and they can be synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, ammonium, and organic alkali salts such as, for example, ethylenediamine, ethanolamine, N,N-dialkylenethanolamine, triethanolamine, glucamine and basic aminoacids salts. Examples of the metallic salts include, for example, sodium, potassium, calcium, magnesium, aluminum and lithium salts.

In a particular embodiment of the first, second and third aspects of the present invention, the compound is N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, or a pharmaceutically acceptable salt thereof, preferably N-[1-amino-3-(3-fluorophenyl)propan-2-yl]-5-chloro-4-(4-chloro-1-methyl-1H-pyrazol-5-yl)thiophene-2-carboxamide hydrochloride, more preferably N-[(2S)-1-amino-3-(3-fluorophenyl)propan-2-yl]-5-chloro-4-(4-chloro-1-methyl-1H-pyrazol-5-yl)thiophene-2-carboxamide hydrochloride.

In another particular embodiment of the first, second and third aspects of the present invention, the compound is [2-(2,6-dichloroanilino)-4,5-dihydroimidazol-1-yl]-phenylmethanone or a pharmaceutically acceptable salt thereof, preferably [2-(2,6-dichloroanilino)-4,5-dihydroimidazol-1-yl]-phenylmethanone.

In another particular embodiment of the first, second and third aspects of the present invention, the compound is 2-amino-5-bromo-4-phenyl-1H-pyrimidin-6-one or a pharmaceutically acceptable salt thereof, preferably 2-amino-5-bromo-4-phenyl-1H-pyrimidin-6-one.

In another particular embodiment of the first, second and third aspects of the present invention, the compound is N-(diaminomethylidene)-2-(2,6-dichlorophenyl)acetamide or a pharmaceutically acceptable salt thereof, preferably N-(diaminomethylidene)-2-(2,6-dichlorophenyl)acetamide hydrochloride.

In another particular embodiment of the first, second and third aspects of the present invention, the compound is 2-(1,4-dioxaspiro[4.5]decan-2-ylmethyl)guanidine or a pharmaceutically acceptable salt thereof, preferably 2-(1,4-dioxaspiro[4.5]decan-2-ylmethyl)guanidine hydroiodide, more preferably 2-(1,4-dioxaspiro[4.5]decan-2-ylmethyl)guanidine sulfate.

In another particular embodiment of the first, second and third aspects of the present invention, the compound is 4-(2-(diaminomethylidene)hydrazinyl)phenyl]iminothiourea or a pharmaceutically acceptable salt thereof, preferably 4-(2-(diaminomethylidene)hydrazinyl)phenyl]iminothiourea.

In another particular embodiment of the first, second and third aspects of the present invention, the compound is 1,2-bis[(4-chlorophenyl)methylideneamino]guanidine preferably the (E) isomer 1,2-bis[(E)-(4-chlorophenyl)methylideneamino]guanidine or a pharmaceutically acceptable salt thereof, preferably 1,2-bis[(4-chlorophenyl)methylideneamino]guanidine hydrochloride, more preferably 1,2-bis[(E)-(4-chlorophenyl)methylideneamino]guanidine hydrochloride.

In another particular embodiment of the first, second and third aspects of the present invention, the compound is N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, or a pharmaceutically acceptable salt thereof, preferably N-[1-(1-naphthyl)ethyl]-3-[3-(trifluoromethyl)phenyl]propan-1-amine hydrochloride, more preferably N-[(1R)-1-(1-naphthyl)ethyl]-3-[3-(trifluoromethyl)phenyl]propan-1-amine hydrochloride.

In another particular embodiment of the first, second and third aspects of the present invention, the compound is methyl N-(6-propoxy-1H-benzimidazol-2-yl) carbamate or a pharmaceutically acceptable salt thereof, preferably methyl N-(6-propoxy-1H-benzimidazol-2-yl) carbamate.

In another particular embodiment of the first, second and third aspects of the present invention, the compound is 5-[2-[[3-(1,3-Benzodioxol-5-yl)-1-methylpropyl]amino]-1-hydroxyethyl]-2-hydroxybenzamide or a pharmaceutically acceptable salt thereof, preferably 5-[2-[[3-(1,3-Benzodioxol-5-yl)-1-methylpropyl]amino]-1-hydroxyethyl]-2-hydroxybenzamide.

In another particular embodiment of the first, second and third aspects of the present invention, the compound is 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, or a pharmaceutically acceptable salt thereof, preferably 2-amino-9-[4-(hydroxymethyl)cyclopent-2-en-1-yl]-1H-purin-6-one, more preferably 2-amino-9-[(1R,4S)-4-(hydroxymethyl)cyclopent-2-en-1-yl]-1H-purin-6-one.

In another particular embodiment of the first, second and third aspects of the present invention, the compound is 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, or a pharmaceutically acceptable salt thereof, preferably 4-[2-amino-6-(cyclopropylamino)-9H-purin-9-yl]-2-cyclopentene-1-methanol sulfate, more preferably (1S,4R)-4-[2-amino-6-(cyclopropylamino)-9H-purin-9-yl]-2-cyclopentene-1-methanol sulfate.

In another particular embodiment of the first, second and third aspects of the present invention, the compound is 4-amino-N-(4-methylpyrimidin-2-yl)benzenesulfonamide or a pharmaceutically acceptable salt thereof, preferably 4-amino-N-(4-methylpyrimidin-2-yl)benzenesulfonamide.

The compounds according to the invention may be administered by any appropriate route (via), such as, oral (e.g., oral, sublingual, etc.), parenteral (e.g., subcutaneous, intramuscular, intravenous, intramuscular, etc.), vaginal, rectal, nasal, topical, ophthalmic, etc., preferably oral or parenteral, more preferably oral.

In particular, the compounds according to the invention are administered as a pharmaceutical composition, which comprises the corresponding (active) compound, and one or more pharmaceutically acceptable excipients.

The compounds according to the invention may be administered in a “therapeutically effective amount”, i.e. a nontoxic but sufficient amount of the corresponding compound to provide the desired effect. The amount that is “effective” will vary from subject to subject, depending on the age and general condition of the individual, the particular compound administered, and the like. Thus, it is not always possible to specify an exact “therapeutically effective amount”. However, an appropriate amount in any individual case may be determined by one of ordinary skill in the art using routine experimentation.

The compounds according to the invention will typically be administered once or more times a day, for example 1, 2, 3 or 4 times daily, with typical total daily doses depending on the particular compound and severity of the disease, and may be easily determined by the skilled practitioner. By way of example, typical total daily doses of 1,2-bis[(4-chlorophenyl)methylideneamino]guanidine, preferably 1,2-bis[(E)-(4-chlorophenyl)methylideneamino]guanidine or a pharmaceutically acceptable salt thereof are in the range of from 0.1 to 3600 mg/day (expressed as 1,2-bis[(4-chlorophenyl)methylideneamino]guanidine free base, preferably 1,2-bis[(E)-(4-chlorophenyl)methylideneamino]guanidine free base), preferably from 1 to 360 mg/day. The compound may be administered by oral route.

Typical total daily doses of N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, or a pharmaceutically acceptable salt thereof are in the range of from 0.1 to 2700 mg/day (expressed as N-[1-(1-naphthyl)ethyl]-3-[3-(trifluoromethyl)phenyl]propan-1-amine free base, preferably N-[(1R)-1-(1-naphthyl)ethyl]-3-[3-(trifluoromethyl)phenyl]propan-1-amine free base), preferably from 1 to 270 mg/day. The compound may be administered by oral route.

Typical total daily doses of N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, or a pharmaceutically acceptable salt thereof administered by oral route are in the range of from 1 to 1800 mg/day (expressed as N-[1-amino-3-(3-fluorophenyl)propan-2-yl]-5-chloro-4-(4-chlorol-methyl-1H-pyrazol-5-yl)thiophene-2-carboxamide free base, preferably as N-[(2S)-1-amino-3-(3-fluorophenyl)propan-2-yl]-5-chloro-4-(4-chloro-1-methyl-1H-pyrazol-5-yl)thiophene-2-carboxamide, free base), preferably from 10 to 175 mg/day.

Typical total daily doses of [2-(2,6-dichloroanilino)-4,5-dihydroimidazol-1-yl]-phenylmethanone or a pharmaceutically acceptable salt thereof are in the range of from 0.1 to 1000 mg/day (expressed as [2-(2,6-dichloroanilino)-4,5-dihydroimidazol-1-yl]-phenylmethanone free base), preferably from 1 to 100 mg/day. The compound may be administered by oral route.

Typical total daily doses of methyl N-(6-propoxy-1H-benzimidazol-2-yl) carbamate or a pharmaceutically acceptable salt thereof are in the range of from 0.1 to 6000 mg/day (expressed as methyl N-(6-propoxy-1H-benzimidazol-2-yl) carbamate free base), preferably from 1 to 600 mg/day, even more preferably from 200 to 600 mg/day. The compound may be administered by oral route.

Typical total daily doses of 4-(2-(diaminomethylidene)hydrazinyl)phenyl]iminothiourea or a pharmaceutically acceptable salt thereof are in the range of from 0.1 to 1200 mg/day (expressed as 4-(2-(diaminomethylidene)hydrazinyl)phenyl]iminothiourea free base), preferably from 1 to 100 mg/day. The compound may be administered by oral route.

Typical total daily doses of N-(diaminomethylidene)-2-(2,6-dichlorophenyl)acetamide or a pharmaceutically acceptable salt thereof are in the range of from 0.1 to 700 mg/day (expressed as N-(diaminomethylidene)-2-(2,6-dichlorophenyl)acetamide free base), preferably from 1 to 7 mg/day. The compound may be administered by oral route.

Typical total daily doses of 2-amino-5-bromo-4-phenyl-1H-pyrimidin-6-one or a pharmaceutically acceptable salt thereof are in the range of from 0.1 to 30000 mg/day (expressed as 2-amino-5-bromo-4-phenyl-1H-pyrimidin-6-one free base), preferably from 1 to 3000 mg/day. The compound may be administered by oral route.

Typical total daily doses of 2-(1,4-dioxaspiro[4.5]decan-2-ylmethyl)guanidine or a pharmaceutically acceptable salt thereof are in the range of from 0.1 to 7500 mg/day (expressed as 2-(1,4-dioxaspiro[4.5]decan-2-ylmethyl)guanidine free base), preferably from 1 to 75 mg/day. The compound may be administered by oral route.

Typical total daily doses of 5-[2-[[3-(1,3-Benzodioxol-5-yl)-1-methylpropyl]amino]-1-hydroxyethyl]-2-hydroxybenzamide or a pharmaceutically acceptable salt thereof are in the range of from 0.1 to 4000 mg/day (expressed as 5-[2-[[3-(1,3-Benzodioxol-5-yl)-1-methylpropyl]amino]-1-hydroxyethyl]-2-hydroxybenzamide free base), preferably from 1 to 400 mg/day. The compound may be administered by oral route.

Typical total daily doses of 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, or a pharmaceutically acceptable salt thereof are in the range of from 0.1 to 6000 mg/day (expressed as 2-amino-9-[4-(hydroxymethyl)cyclopent-2-en-1-yl]-1H-purin-6-one free base, preferably as 2-amino-9-[(1R,4S)-4-(hydroxymethyl)cyclopent-2-en-1-yl]-1H-purin-6-one free base), preferably from 1 to 600 mg/day. The compound may be administered by oral route or intravenously.

Typical total daily doses of 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, or a pharmaceutically acceptable salt thereof are in the range of from 0.1 to 6000 mg/day (expressed as 4-[2-amino-6-(cyclopropylamino)-9H-purin-9-yl]-2-cyclopentene-1-methanol free base, preferably as (1S,4R)-4-[2-amino-6-(cyclopropylamino)-9H-purin-9-yl]-2-cyclopentene-1-methanol free base), preferably from 1 to 600 mg/day. The compound may be administered by oral route.

Typical total daily doses of 4-amino-N-(4-methylpyrimidin-2-yl)benzenesulfonamide or a pharmaceutically acceptable salt thereof are in the range of from 1 to 14000 mg/day (expressed as 4-amino-N-(4-methylpyrimidin-2-yl)benzenesulfonamide free base), preferably from 10 to 1400 mg/day. The compound may be administered by oral route.

The term “subject” refers to a mammal, preferably a human.

In the fourth aspect, the present invention relates to a combination comprising one or more compounds selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-03, PD-01, LuAF-82422, NPT-088, UB-312, and Anle138b or a pharmaceutically acceptable salt thereof, for use in the treatment and/or prevention of synucleinopathies, with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism and wherein at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof.

In the fifth aspect the invention relates to the use of a combination comprising one or more compounds selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-03, PD-01, LuAF-82422, NPT-088, UB-312, and Anle138b or a pharmaceutically acceptable salt thereof, in the manufacture of a medicament for the treatment and/or prevention of synucleinopathies, with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism and wherein at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide or a pharmaceutically acceptable salt thereof.

In a sixth aspect the invention relates to a method of treating and/or preventing of synucleinopathies in a subject, comprising administering to said subject a therapeutically effective amount of a combination comprising one or more compounds selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-03, PD-01, LuAF-82422, NPT-088, UB-312, and Anle138b or a pharmaceutically acceptable salt thereof, with the proviso that when the compound is cinacalcet (i.e. N-[(1R)-1-(1-naphthyl)ethyl]-3-[3-(trifluoromethyl)phenyl]propan-1-amine) the treatment and/or prevention are not carried out in subjects having an abnormal calcium metabolism such as hypercalcemia or hyperparathyroidism and wherein at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide, 2-(1,4-dioxaspiro[4.5]decan-2-ylmethyl)guanidine, 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof.

In an embodiment of the fourth, fifth and sixth aspects of the invention, at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide and 2-(1,4-dioxaspiro[4.5]decan-2-ylmethyl)guanidine, or a pharmaceutically acceptable salt thereof.

In another embodiment of the fourth, fifth and sixth aspects of the invention, at least one of the compounds in the combination is selected from the group consisting of 1,2-bis[(4-chlorophenyl)methylideneamino]guanidine, N-[(1R)-1-(1-naphthyl)ethyl]-3-[3-(trifluoromethyl)phenyl]propan-1-amine, methyl N-(6-propoxy-1H-benzimidazol-2-yl) carbamate, [4-(2-(diaminomethylidene)hydrazinyl)phenyl]iminothiourea, 5-[2-[[3-(1,3-benzodioxol-5-yl)-1-methylpropyl]amino]-1-hydroxyethyl]-2-hydroxybenzamide, 2-amino-9-[(1R,4S)-4-(hydroxymethyl)cyclopent-2-en-1-yl]-1H-purin-6-one, (1S,4R)-4-[2-amino-6-(cyclopropylamino)-9H-purin-9-yl]-2-cyclopentene-1-methanol and 4-amino-N-(4-methylpyrimidin-2-yl)benzenesulfonamide, or a pharmaceutically acceptable salt thereof.

In a particular embodiment of the fourth, fifth and sixth aspects mentioned above, in which one of the compounds of the combination is N-[(1R)-1-(1-naphthyl)ethyl]-3-[3-(trifluoromethyl)phenyl]propan-1-amine, or a pharmaceutically acceptable salt thereof, the synucleinopathy is not associated with an abnormal calcium metabolism, such as hypercalcemia, or hyperparathyroidism.

In a particular embodiment of the fourth, fifth and sixth aspects mentioned above, the synucleinopathy to be treated and/or prevented is selected from the group consisting of Parkinson's disease (PD), Multiple System Atrophy (MSA) and Dementia with Lewy Bodies. Preferably, the synucleinopathy to be treated and/or prevented is Parkinson's disease (PD).

The term “combination” refers to a product comprising one or more of the defined compounds, either in a single composition or in several compositions (or units), in which case the corresponding compounds are distributed among the several compositions. Preferably, the combination refers to several compositions, in particular comprising one composition (or unit) per compound (compound as defined above) of the combination. The expression “one or more” when characterizing the combination refers to at least one, preferably 1, 2, 3, 4, or 5 compounds, more preferably, 1, 2 or 3 compounds, even more preferably 1 or 2 compounds.

When the combination is in the form of a single composition, the compounds present in the combination are always administered simultaneously.

When the combination is in the form of several compositions (or units), each of them having at least one of the compounds of the combination, the compositions or (units) may be administered simultaneously, sequentially or separately.

Simultaneous administration means that the compounds or compositions (or units) are administered at the same time.

Sequential administration means that the compounds or compositions (or units) are administered at different time points in a chronologically staggered manner.

Separate administration means that the compounds or compositions (or units) are administered at different time points independently of each other.

1,2-bis[(4-chlorophenyl)methylideneamino]guanidine, N-[1-(1-naphthyl)ethyl]-3-[3-(trifluoromethyl)phenyl]propan-1-amine, N-[1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, 4-[2-amino-6-(cyclopropylamino)-9H-purin-9-yl]-2-cyclopentene-1-methanol, 4-amino-N-(4-methylpyrimidin-2-yl)benzenesulfonamide including their isomers, and/or pharmaceutically acceptable salts have been described in detail above.

Levodopa, commonly known as L-DOPA or via its chemical name (S)-2-amino-3-(3,4-dihydroxyphenyl)propanoic acid has the chemical structure depicted below.

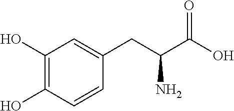 [SMILES: N[C@@H](Cc1ccc(O)c(O)c1)C(=O)O]

This compound is a precursor of dopamine, being converted in the body to the latter, through decarboxylation. It is used in the clinical treatment of PD and is commercially available.

Carbidopa, or (2S)-3-(3,4-dihydroxyphenyl)-2-hydrazino-2-methylpropanoic acid has the structure depicted below.

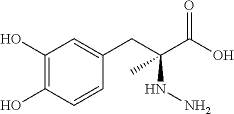 [SMILES: C[C@@](Cc1ccc(O)c(O)c1)(NN)C(=O)O]

This compound inhibits the conversion of levodopa to dopamine, outside of the brain, by inhibiting the activity of aromatic-L-amino-acid decarboxylase, thus preventing the peripheral metabolism of levodopa, resulting in an increased availability of levodopa in the central nervous system. This compound is used in the clinical treatment of PD and is commercially available.

Benserazide, or 2-amino-3-hydroxy-N-(2,3,4-trihydroxybenzyl)propane hydrazide, has the structure depicted below.

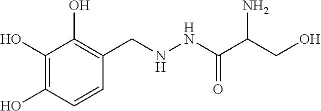 [SMILES: NC(CO)C(=O)NNCc1ccc(O)c(O)c1O]

This compound has a mechanism of action similar to carbidopa, inhibits the conversion of levodopa to dopamine, preventing the peripheral metabolism of levodopa. This compound is used in the clinical treatment of PD and is commercially available.

Entacapone, or (2E)-2-cyano-3-(3,4-dihydroxy-5-nitrophenyl)-N,N-diethylprop-2-enamide, and opicapone or 5-[3-(2,5-Dichloro-4,6-dimethyl-1-oxido-3-pyridinyl)-1,2,4-oxadiazol-5-yl]-3-nitro-1,2-benzenediol have the structure depicted below, left and right respectively.

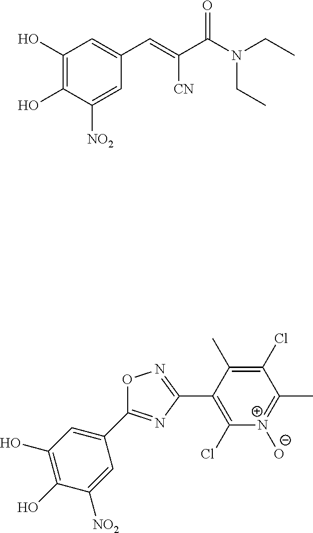 [SMILES: CCN(CC)C(=O)/C(C#N)=C/c1cc(O)c(O)c([N+](=O)[O-])c1 ; Cc1c(Cl)c(C)[n+]([O-])c(Cl)c1-c1noc(-c2cc(O)c(O)c([N+](=O)[O-])c2)n1]

These compounds are used in combination therapy of PD and act by inhibiting catechol-O-methyltransferase, leading to increased levels of levodopa in the brain. Both are commercially available.

Amantadine, or adamantan-1-amine, has the chemical structure depicted below.

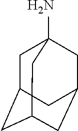 [SMILES: NC12CC3CC(CC(C3)C1)C2]

This compound is recommended for use in combination therapy with levodopa, to prevent and/or mitigate its collateral effects, and it is commercially available.

Pimavanserin, or N-(4-fluorophenylmethyl)-N-(1-methylpiperidin-4-yl)-N′-(4-(2-methylpropyloxy)phenylmethyl)carbamide, has the chemical structure depicted below.

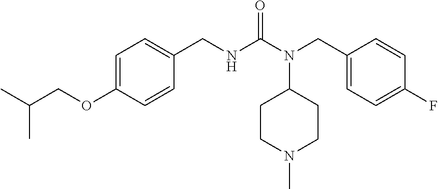 [SMILES: CC(C)COc1ccc(CNC(=O)N(Cc2ccc(F)cc2)C2CCN(C)CC2)cc1]

This compound has been approved for the treatment of PD, and is currently being investigated as an anti-psychotic agent, and it is commercially available.

Rotigotine, or (S)-6-[Propyl(2-thiophen-2-ylethyl)amino]-5,6,7,8-tetrahydro-naphthalen-1-ol, has the chemical structure depicted below.

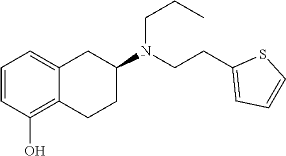 [SMILES: CCCN(CCc1cccs1)[C@H]1CCc2c(O)cccc2C1]

This compound is a dopamine agonist, and is currently in clinical use for the treatment of PD. Additionally, it also possesses antidepressant effects, and it has been further approved for the treatment of restless legs syndrome. This compound is commercially available.

Ropinirole, or 4-[2-(dipropylamino)ethyl]-1,3-dihydro-2H-indol-2-one, has the chemical structure depicted below.

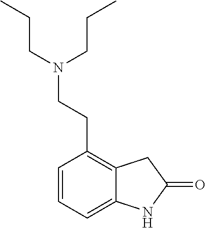 [SMILES: CCCN(CCC)CCc1cccc2c1CC(=O)N2]

Similarly to rotigotine this compound is also a dopamine receptor agonist, and has been used for the treatment of both PD and restless legs syndrome. It is commercially available.

Apomorphine, having the chemical name of (6aR)-6-methyl-5,6,6a,7-tetrahydro-4H-dibenzo[de,g]quinoline-10,11-diol, has the chemical structure depicted below.

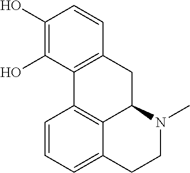 [SMILES: CN1CCc2cccc3c2[C@H]1Cc1ccc(O)c(O)c1-3]

This compound is a derivative of morphine, and exhibits the catechol moiety, similar to that of dopamine. It is an agonist of both D₁ and D2 dopamine receptors, and has been developed for the treatment of advanced PD, wherein there is a reduced response to standard anti-Parkinson drugs, such as levodopa. It is commercially available.

Rasagiline, having the chemical name of (R)—N-(prop-2-ynyl)-2,3-dihydro-1H-inden-1-amine, has the chemical structure depicted below.

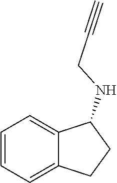

This compound is an irreversible MAO-B inhibitor and has been developed for the symptomatic treatment of early Parkinson's disease, being especially useful in managing the non-motor symptoms. It is commercially available.

Safinamide, or (2S)-2-[[4-[(3-fluorophenyl)methoxy]phenyl]methylamino]propanamide, has the chemical structure depicted below.

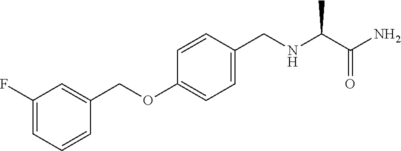 [SMILES: C[C@H](NCc1ccc(OCc2cccc(F)c2)cc1)C(N)=O]

This compound is also an MAO-B inhibitor and has been developed for use in patients taking levodopa/carbidopa to reduce “off-phases” wherein the symptoms do not respond to the medication. It is commercially available.

Istradefylline, or 8-[(E)-2-(3,4-dimethoxyphenyl)vinyl]-1,3-diethyl-7-methyl-3,7-dihydro-1H-purine-2,6-dione, has the chemical structure depicted below.

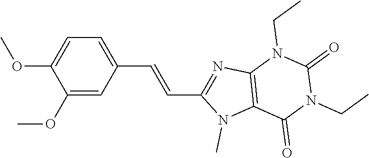 [SMILES: CCn1c(=O)c2c(nc(/C=C/c3ccc(OC)c(OC)c3)n2C)n(CC)c1=O]

This compound has been developed to reduce “off-phases” during PD treatment with levodopa, wherein the symptoms do not respond to the medication. It is commercially available.

KW-6356, is a compound ongoing clinical development by Kyowa Kirin, for the treatment of PD. It is an adenosine A2A-receptor antagonist.

Donepezil, having the chemical name 2-[(1-Benzyl-4-piperidyl)methyl]-5,6-dimethoxy-2,3-dihydroinden-1-one, has the chemical structure depicted below.

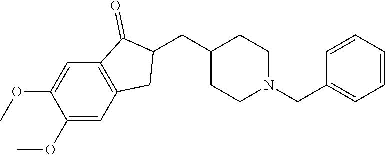 [SMILES: COc1cc2c(cc1OC)C(=O)C(CC1CCN(Cc3ccccc3)CC1)C2]

This compound is a cholinesterase inhibitor, and has been developed for the treatment of dementia in Alzheimer's disease, being also useful for the treatment of PD dementia. It is commercially available, and it is used as the racemate.

Rivastigmine, or (S)-3-[1-(dimethylamino)ethyl]phenyl N-ethyl-N-methylcarbamate, has the chemical structure depicted below.

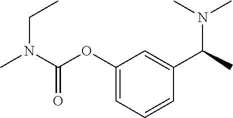

This compound is an acetylcholinesterase inhibitor, and has been developed for the treatment of mild to moderate dementia in both Alzheimer's and Parkinson's diseases. It is commercially available.

Liraglutide is an acylated glucagon-like peptide-1, which has been developed for the treatment of diabetes-type II and obesity. It has been shown to have neuroprotective effects in mice models of PD, while its usefulness for the treatment of PD is currently being evaluated in clinical trials. It is commercially available.

Prasinezumab is a humanized monoclonal antibody targeting α-synuclein, developed by Roche, and currently ongoing phase II clinical trials, to assess its usefulness for the treatment of PD.

Venglustat, also known as ibiglustat, having the chemical name (3S)-1-azabicyclo[2.2.2]octan-3-yl N-{2-[2-(4-fluorophenyl)-1,3-thiazol-4-yl]propan-2-yl}carbamate, has the chemical structure depicted below.

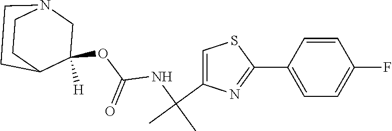 [SMILES: CC(C)(NC(=O)O[C@@H]1CN2CCC1CC2)c1csc(-c2ccc(F)cc2)n1]

This compound has been developed by Sanofi Genzyme, as a glucosylceramide synthase inhibitor, for the treatment of Fabry, Gaucher and Parkinson's diseases. It is commercially available.

Dipraglurant, also known 6-fluoro-2-[4-(2-pyridinyl)-3-butyn-1-yl]imidazo[1,2-a]pyridine, has the chemical structure depicted below.

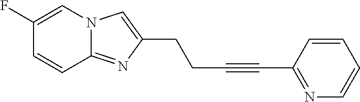 [SMILES: Fc1ccc2nc(CCC#Cc3ccccn3)cn2c1]

This compound is a negative allosteric modulator of the metabotropic glutamate receptor 5, and it is being developed by Addex Therapeutics for the treatment of Parkinson's disease and is commercially available.

Mesdopetam, or N-{2-[3-fluoro-5-(methanesulfonyl)phenoxy]ethyl}propan-1-amine, has the chemical structure depicted below.

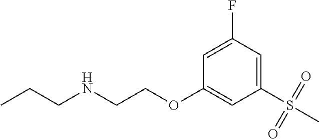 [SMILES: CCCNCCOc1cc(F)cc(S(C)(=O)=O)c1]

This compound is D₃-dopamine blocker, currently in development by IRLAB Therapeutics AB (phase II clinical trials), for the treatment of PD. It is commercially available or can be synthesized according to WO 2012/143337.

SEP-363856, also shortened to SEP-856, having the chemical name (S)-1-(4,7-dihydro-5H-thieno[2,3-c]pyran-7-yl)-N-methylmethanamine, has the chemical structure depicted below.

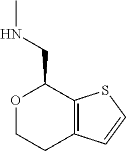

This compound is currently being developed for the treatment of schizophrenia and PD psychosis. This compound can be prepared according to EP 2507245 B1.

Cinpanemab (BIIB-054) is a monoclonal antibody targeting pathological α-synuclein aggregates, developed by Biogen Inc, and currently ongoing phase II clinical trials, to assess its usefulness for the treatment of PD.

Posiphen (ANVS-401), also known as (+)-phenserine, having the chemical name (3aR,8aS)-1,3a,8-trimethyl-1H,2H,3H,3aH,8H,8aH-pyrrolo[2,3-b]indol-5-yl N-phenylcarbamate has the chemical structure depicted below.

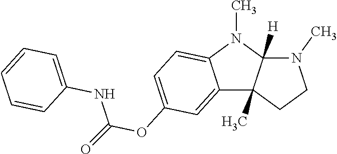 [SMILES: CN1CC[C@]2(C)c3cc(OC(=O)Nc4ccccc4)ccc3N(C)[C@H]12]

This compound is under development for the treatment of Alzheimer's disease, Parkinson's disease and dementia in Down' Syndrome. It works by inhibiting the synthesis of α-synuclein, tau and APP, currently in development by Annovis Bio Inc and in Phase I clinical trials for PD.

MEDI-1341 (TAK-341) is a monoclonal antibody targeting α-synuclein, developed by AstraZeneca, and currently ongoing phase I clinical trials to assess its usefulness for the treatment of PD.

UCB-0599 is a small molecule α-synuclein inhibitor that is currently in development (Phase I) by UCB SA for the treatment of PD.

ABBV-0805 (BAN-0805) is a monoclonal antibody targeting α-synuclein, developed by AbbVie Inc, and currently ongoing phase I clinical trials to assess its usefulness for the treatment of PD.

NPT-20011 is a small molecule α-synuclein aggregation inhibitor that is currently in development (Phase I) by Neuropore Therapies for the treatment of PD and MSA.

PD-01 and PD-03 are peptide vaccines that target α-synuclein and are currently in development (Phase I) by AFFiRis AG for the treatment of PD.

LuAF-82422 is a monoclonal antibody targeting α-synuclein, developed by Genmab AS, and currently ongoing phase I clinical trials to assess its usefulness for the treatment of PD.

NPT-088 is an Ig fusion protein that acts by targeting amyloid beta, α-synuclein, prion and tau protein. It is developed by Proclara Biosciences Inc, and currently ongoing phase I clinical trials to assess its usefulness for the treatment of Alzheimer's disease and PD.

UB-312 is an α-synuclein vaccine, developed by United Neuroscience Ltd, and currently ongoing phase I clinical trials in PD and MSA.

Anle138b, having the chemical name 5-(1,3-benzodioxol-5-yl)-3-(3-bromophenyl)-1H-pyrazole, has the chemical structure depicted below.

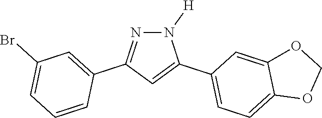 [SMILES: Brc1cccc(-c2cc(-c3ccc4c(c3)OCO4)[nH]n2)c1]

This compound is an α-synuclein aggregation inhibitor that is currently in clinical trials phase I for PD, MSA, Alzheimer's disease and prion disease.

In a preferred embodiment of the fourth, fifth and sixth aspect of the present invention, the combination comprises N-[(2S)-1-amino-3-(3-fluorophenyl)propan-2-yl]-5-chloro-4-(4-chloro-1-methyl-1H-pyrazol-5-yl)thiophene-2-carboxamide and one or more (preferably one) compounds selected from N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In another preferred embodiment, the combination comprises at least [2-(2,6-dichloroanilino)-4,5-dihydroimidazol-1-yl]-phenylmethanone or a pharmaceutically acceptable salt thereof (such as the hydrochloride) and one or more (preferably one) compounds selected from 1,2-bis[(4-chlorophenyl)methylideneamino]guanidine, preferably 1,2-bis[(E)-(4-chlorophenyl)methylideneamino]guanidine, N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In another preferred embodiment of the fourth, fifth and sixth aspect of the present invention, the combination comprises 2-amino-5-bromo-4-phenyl-1H-pyrimidin-6-one or a pharmaceutically acceptable salt thereof (such as the hydrochloride) and one or more (preferably one) compounds selected from 1,2-bis[(4-chlorophenyl)methylideneamino]guanidine, preferably 1,2-bis[(E)-(4-chlorophenyl)methylideneamino]guanidine, N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In another preferred embodiment of the fourth, fifth and sixth aspect of the present invention, the combination comprises N-(diaminomethylidene)-2-(2,6-dichlorophenyl)acetamide or a pharmaceutically acceptable salt thereof (preferably hydrochloride) and one or more (preferably one) compounds selected from 1,2-bis[(4-chlorophenyl)methylideneamino]guanidine, preferably 1,2-bis[(E)-(4-chlorophenyl)methylideneamino]guanidine, N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-]H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In another preferred embodiment, the combination comprises 2-(1,4-dioxaspiro[4.5]decan-2-ylmethyl)guanidine or a pharmaceutically acceptable salt thereof (preferably hydroiodide or sulfate) and one or more (preferably one) compounds selected from N-[1-(1-naphthyl)ethyl]-3-[3-(trifluoromethyl)phenyl]propan-1-amine, preferably 1,2-bis[(E)-(4-chlorophenyl)methylideneamino]guanidine, N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In another embodiment, the combination comprises 4-(2-(diaminomethylidene)hydrazinyl)phenyl]iminothiourea or a pharmaceutically acceptable salt thereof (preferably free base) and one or more (preferably one) compounds selected from 1,2-bis[(4-chlorophenyl)methylideneamino]guanidine, preferably 1,2-bis[(E)-(4-chlorophenyl)methylideneamino]guanidine, N-[1-(1-naphthyl)ethyl]-3-[3-(trifluoromethyl)phenyl]propan-1-amine, preferably N-[(1R)-1-(1-naphthyl)ethyl]-3-[3-(trifluoromethyl)phenyl]propan-1-amine, N-[1-amino-3-(3-fluorophenyl)propan-2-yl]-5-chloro-4-(4-chloro-1-methyl-1H-pyrazol-5-yl)thiophene-2-carboxamide, preferably N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, N-(diaminomethylidene)-2-(2,6-dichlorophenyl)acetamide, 2-amino-5-bromo-4-phenyl-]H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, preferably 2-amino-9-[(1R,4S)-4-(hydroxymethyl)cyclopent-2-en-1-yl]-1H-purin-6-one, 4-[2-amino-6-(cyclopropylamino)-9H-purin-9-yl]-2-cyclopentene-1-methanol, preferably (1S,4R)-4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-01, PD-03, LuAF-82422, NPT-088, UB-312, and Anle138b, or a pharmaceutically acceptable salt thereof.

In particular, the combinations according to the invention are administered as pharmaceutical compositions, which comprise the corresponding (active) compounds and a pharmaceutically acceptable excipient, as previously defined.

The combinations according to the invention will typically be administered one or more times a day, for example 1, 2, 3 or 4 times daily, with typical total daily doses depending on the particular compound and severity of the disease, and may be easily determined by the skilled practitioner.

In a seventh aspect, the present invention refers to pharmaceutical compositions comprising a compound according to the first, second or third aspects, and/or a combination according to the fourth, fifth or sixth aspect, for use in the treatment and/or prevention of synucleinopathies, with the proviso that, when the compound is cinacalcet (i.e. N-[(1R)-1-(1-naphthyl)ethyl]-3-[3-(trifluoromethyl)phenyl]propan-1-amine), the treatment and/or prevention are not carried out in subjects having an abnormal calcium metabolism such as hypercalcemia or hyperparathyroidism.

In a particular embodiment, a pharmaceutical composition according to the seventh aspect further comprises one or more pharmaceutically acceptable excipients.

In a particular embodiment of the seventh aspect, the synucleinopathy to be treated and/or prevented is selected from the group consisting of Parkinson's disease (PD), Multiple System Atrophy (MSA) and Dementia with Lewy Bodies. Preferably, the synucleinopathy to be treated and/or prevented is Parkinson's disease (PD).

The term “pharmaceutically acceptable excipient” refers to a vehicle, diluent, or adjuvant that is administered with the active ingredient. Such pharmaceutical excipients can be sterile liquids, such as water and oils, including those of petroleum, animal, vegetable, or synthetic origin, such as peanut oil, soybean oil, mineral oil, sesame oil, and the like. Water or saline aqueous solutions and aqueous dextrose and glycerol solutions, particularly for injectable solutions, are preferably used as vehicles. Suitable pharmaceutical vehicles are known by the skilled person.

The pharmaceutically acceptable excipient necessary to manufacture the desired pharmaceutical composition of the invention will depend, among other factors, on the elected administration route. The pharmaceutical compositions may be prepared using standard methods such as those described or referred to in the European and US Pharmacopoeias and similar reference texts.

The following examples represent specific embodiments of the present invention. They do not intend to limit in any way the scope of the invention defined in the present description.

### BRIEF DESCRIPTION OF THE FIGURES

FIG. 1A: shows α-synuclein aggregation kinetics measured by Thioflavin T fluorescence emission, represented as normalised means. Error bars are represented as standard error. Aggregation kinetics in control conditions (filled circle) or in the presence of the tested compounds: Robenidine (filled square); oxibendazole (triangle); guanfacine (inverted triangle); bropirimine (diamond); cinacalcet (empty circle) and ambazone (empty square).

FIG. 1B: shows α-synuclein aggregation kinetics measured by Thioflavin T fluorescence emission, represented as normalised means. Error bars are represented as standard error. Aggregation kinetics in control conditions (filled circle) or in the presence of the tested compounds: medroxalol (filled square); benclonidine (triangle); sulfamerazine (inverted triangle); afuresertib (diamond); (−)-carbovir (empty circle); guanadrel (empty square).

FIG. 2A: shows α-synuclein aggregation measured by light scattering at end-point (48 h). Light-scattering measurements at 300 and 340 nm in control conditions and in the presence of the tested compounds: Robenidine; oxibendazole; guanfacine; bropirimine; cinacalcet, and ambazone. Data is presented as mean and standard error.

FIG. 2B shows α-synuclein aggregation measured by light scattering at end-point (48 h). Light-scattering measurements at 300 and 340 nm in control conditions and in the presence of the tested compounds: medroxalol; benclonidine; sulfamerazine; afuresertib; (−)-carbovir; guanadrel. Data is presented as mean and standard error.

FIG. 3-9: show α-synuclein aggregation measured by Transmission Electron Microscopy (TEM) at end-point (48 h). Representative TEM images of α-synuclein in the absence (FIG. 3 control) or presence of the tested compounds: FIG. 4 (Robenidine); FIG. 5 (oxibendazole); FIG. 6 (guanfacine); FIG. 7 (bropirimine); FIG. 8 (cinacalcet) and FIG. 9 (ambazone).

FIG. 10-15: show α-synuclein aggregation measured by TEM at end-point (48 h). Representative TEM images of α-synuclein in the absence (FIG. 10 control) or presence of the tested compounds: FIG. 11 (benclonidine); FIG. 12 (sulfamerazine); FIG. 13 (afuresertib); FIG. 14 ((−)-carbovir); FIG. 15 (guanadrel).

FIG. 16-20 show neuroprotection in MPP+ treated dopaminergic neurons achieved with Guanfacine (16), Bropirimine (17), Benclonidine (18), Afuresertib (19) and Guanadrel (20).

### EXAMPLES

### Materials and Methods

### A. Thioflavin-T Assay

Lyophilized α-synuclein was dissolved in PBS (phosphate buffer solution) to a final solution of 210 μM and filtered through a Millipore 0.22 μm filter. α-synuclein aggregation was performed in a 96-well plate (non-treated, black plastic), each well containing a Teflon® polyball ⅛″ diameter (Polysciences Europe GmbH, Eppelheim, Germany), 40 μM Thioflavin-T (Th-T), 70 μM α-synuclein, 100 μM of the compounds to be tested and PBS up to a final volume of 150 μL. The plates were fixed into an orbital culture shaker Max-Q 4000 (Thermo Scientific, Waltham, Mass., USA) to keep the incubation at 37° C., 100 rpm. Every, 2 h, the fluorescence intensity was measured using a Victor 3.0 Multilabel Reader (PerkinElmer, Waltham, Mass., USA), by exciting the mixtures with 430-450 nm filter and collecting the emission intensity with 480-510 nm filter. Each measurement was performed in triplicates.

Each plate contained three (3) α-synuclein controls in the absence of any of the tested compounds. The averaged Th-T fluorescence obtained for these wells at the end of the experience was normalized to 1, and the kinetic curves in the different wells re-scaled accordingly. After compiling the fluorescence signals, means and standard error of mean (SEM) were used to fit the aggregation kinetic with equation (1),

$$α = 1 - \frac{1}{{k}_{b} ( {e}^{k a t} - 1 ) + 1} \qquad Equation ( 1 )$$

wherein k^{b} and k^{a} indicate the homogeneous nucleation rate constant and the secondary rate constant, accounting for fibril elongation and secondary nucleation, respectively.

Re-scaled curves were used to compare the controls with the effect of the compounds and to ensure that the controls were reproducible between different experiments.

### Light Scattering

Total aggregate formation at 48 h was measured by light scattering adding 80 μL of pre-aggregated α-synuclein into a quartz cuvette. Samples were previously re-suspended by carefully vortexing and pipetting, and then excited at 300 and 340 nm and 90° scattering collected between 280 to 360 nm. These measurements were performed in a Cary Eclipse Fluorescence Spectrophotometer (Agilent, Santa Clara, Calif., USA).

### Transmission Electron Microscopy (TEM)

α-synuclein samples were diluted 1:10 in PBS, sonicated for 5 minutes and 5 μL of the resulting mixture immediately placed on a carbon-coated copper grid. After 5 minutes, samples were carefully dried with a piece of filter paper to remove the excess of liquid and washed with MilliQ water twice. Then, 5 μL of a 2% (w/v) solution of uranyl acetate was placed on top of the grid for 2 minutes. Uranyl acetate excess was removed with filter paper. Finally, grids were left to air-dry for 10 min. Images were obtained using a TEM microscope Jeol 1400 (Peabody, Mass., USA) operating at an accelerating voltage of 120 kV. A minimum of 30 fields were screened for each sample to obtain representative images.

### Test Compounds

Robenidine hydrochloride, cinacalcet hydrochloride, afuresertib hydrochloride, oxibendazole, guanfacine hydrochloride, and sulfamerazine were obtained from MedChemExpress. Benclonidine was obtained from Angene. Ambazone, and (−)-carbovir were obtained from Toronto Research Chemicals. Bropirimine, and guanadrel hydroiodide were obtained from Enamine. Medroxalol was obtained from Coompo.

### Results

TABLE 1
Parameters of alpha-synuclein aggregation kinetics
measured by Thioflavin fluorescence.

| | Kb | Ka |
|---|---|---|
| Control | 0.049 ± 0.018 | 0.183 ± 0.027 |
| Robenidine | 0.001 ± 0.444 | 0.053 ± 0.124 |
| Oxibendazole | 0.003 ± 0.004 | 0.369 ± 0.083 |
| Guanfacine | 0.014 ± 0.005 | 0.161 ± 0.027 |
| Bropirimine | 0.027 ± 0.006 | 0.205 ± 0.015 |
| Cinacalcet | 0.066 ± 0.029 | 0.142 ± 0.032 |
| Ambazone | nd | nd |
| Medroxalol | 0.010 ± 0.005 | 0.312 ± 0.034 |
| Benclonidine | 0.007 ± 0.003 | 0.225 ± 0.024 |
| Sulfamerazine | 0.002 ± 0.001 | 0.248 ± 0.019 |
| Afuresertib | 0.056 ± 0.018 | 0.141 ± 0.022 |
| (−)-Carbovir | 0.027 ± 0.004 | 0.172 ± 0.010 |
| Guanadrel | 0.044 ± 0.008 | 0.150 ± 0.013 |

Kb: homogeneus nucleation rate constant (fibril elongation).
Ka: secondary rate constant (secondary nucleation)

TABLE 2
Normalized light scattering values at 300 and 340 nm

| | 300 nm | 340 nm |
|---|---|---|
| Control | 1 ± 0.22 | 1 ± 0.22 |
| Robenidine | 0.31 ± 0.03 | 0.31 ± 0.02 |
| Oxibendazole | 0.13 ± 0.02 | 0.51 ± 0.10 |
| Guanfacine | 0.65 ± 0.10 | 0.68 ± 0.10 |
| Bropirimine | 0.55 ± 0.07 | 0.79 ± 0.10 |
| Cinacalcet | 0.44 ± 0.17 | 0.48 ± 0.18 |
| Ambazone | 0.61 ± 0.15 | 0.53 ± 0.15 |
| Medroxalol | 0.72 ± 0.03 | 0.75 ± 0.04 |
| Benclonidine | 0.95 ± 0.07 | 0.98 ± 0.07 |
| Sulfamerazine | 0.55 ± 0.10 | 0.78 ± 0.14 |
| Afuresertib | 0.44 ± 0.08 | 0.62 ± 0.09 |
| (−)-Carbovir | 0.72 ± 0.08 | 0.77 ± 0.09 |
| Guanadrel | 0.68 ± 0.06 | 0.68 ± 0.06 |

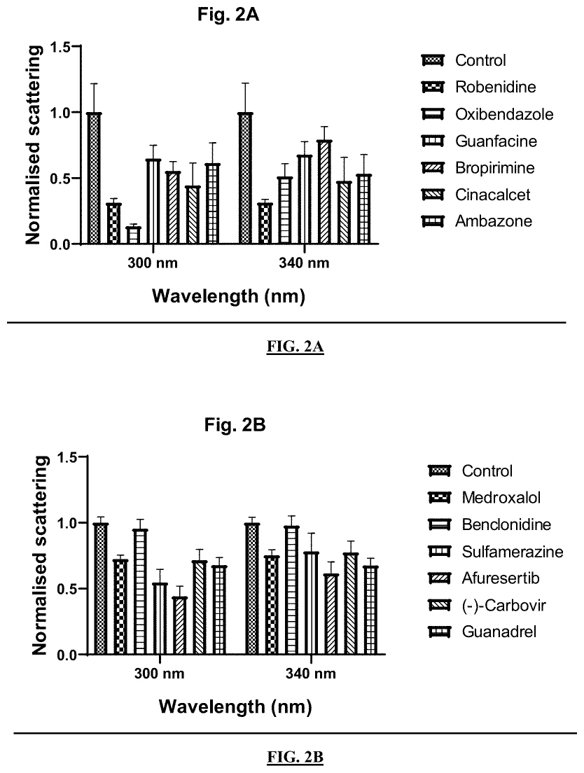

These data are represented in FIGS. 2A and 2B.

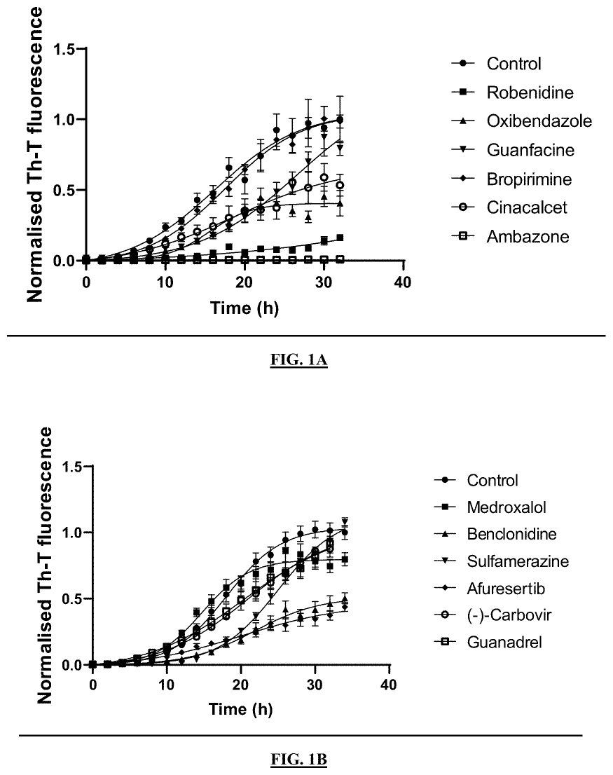

The high-throughput screening protocol uses thioflavin-T (Th-T) as reporter of amyloid formation, completing highly reproducible reactions in less than 48 h. Each compound was tested in 3 independent experiments. Robenidine, oxibendazole and cinacalcet (FIG. 1A) exhibited interesting inhibitory capacity, reducing Th-T-positive species more than 50%. Ambazone interfered with the Th-T fluorescence readout, as it presented significant absorbance at 425 nm. A more modest inhibition was observed with guanfacine. Bropirimine did not cause detectable inhibition of Th-T fluorescence, but it caused a significant effect on aggregation measured by light scattering (see below, FIG. 2A). This apparent discrepancy may be caused by a selective effect of bropirimine on amorphous (not amyloid fibril) aggregates. Other potent inhibitors were benclonidine, afuresertib, and sulfamerazine (FIG. 1B). Other compounds presented more modest inhibition of fibril formation, such as medroxalol, (−)-carbovir, and guanadrel (FIG. 1B).

Light scattering measurements at end-point confirmed the inhibitory effect of the compounds, with significant reduction of the absorbance at 300 and 340 nm by robenidine, oxibendazole, guanfacine, bropirimine, cinacalcet, and ambazone (FIG. 2A). Additional light scattering measurements also confirmed the inhibitory activity of medroxalol, sulfamerazine, afuresertib, (−)-carbovir and guanadrel, that also reduced absorbance at both length waves (FIG. 2B).

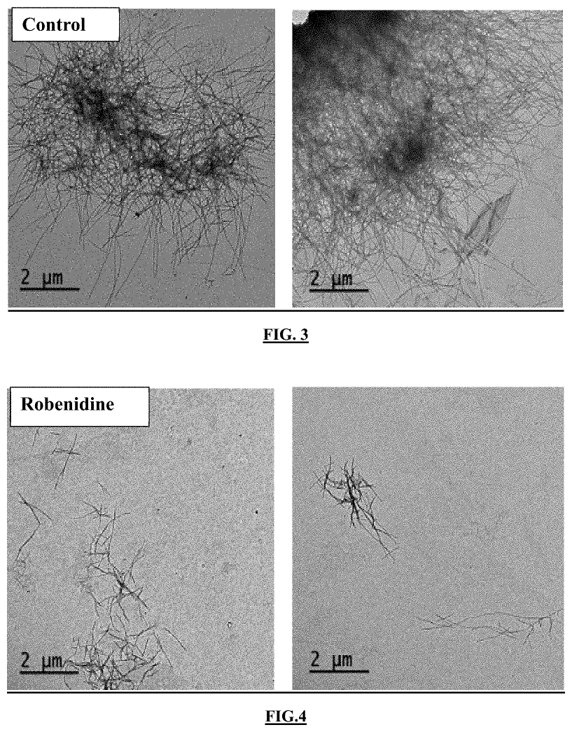

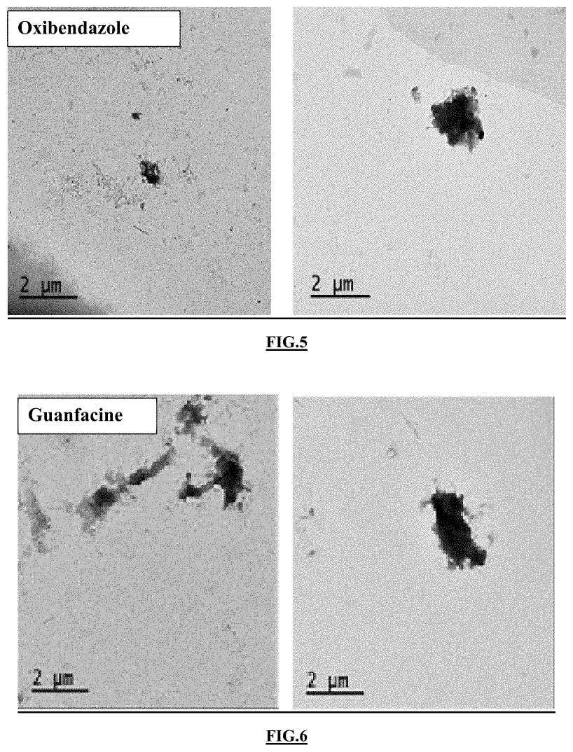

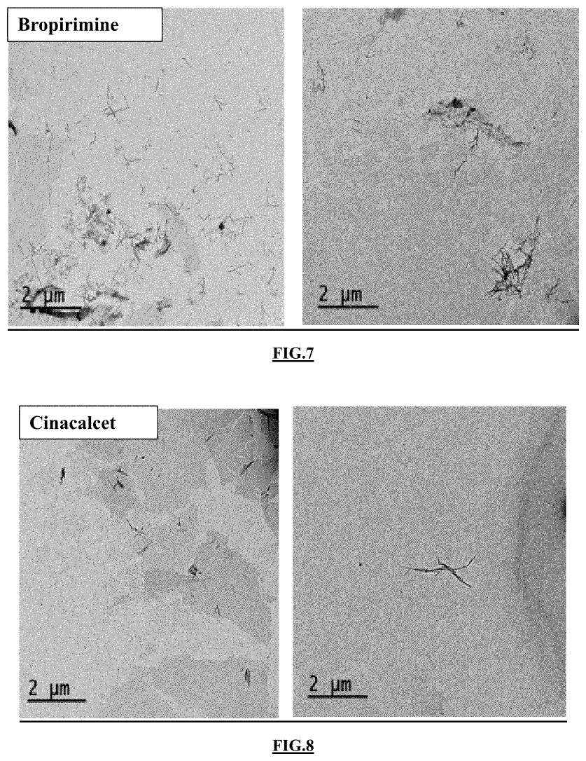

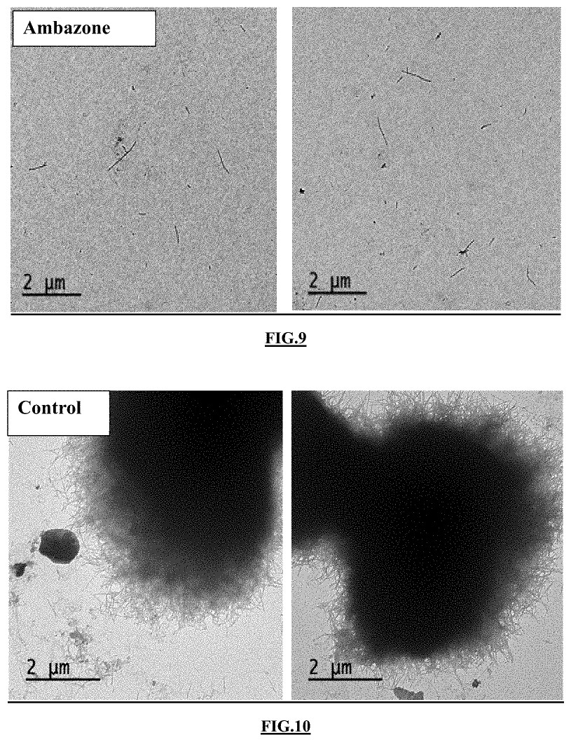

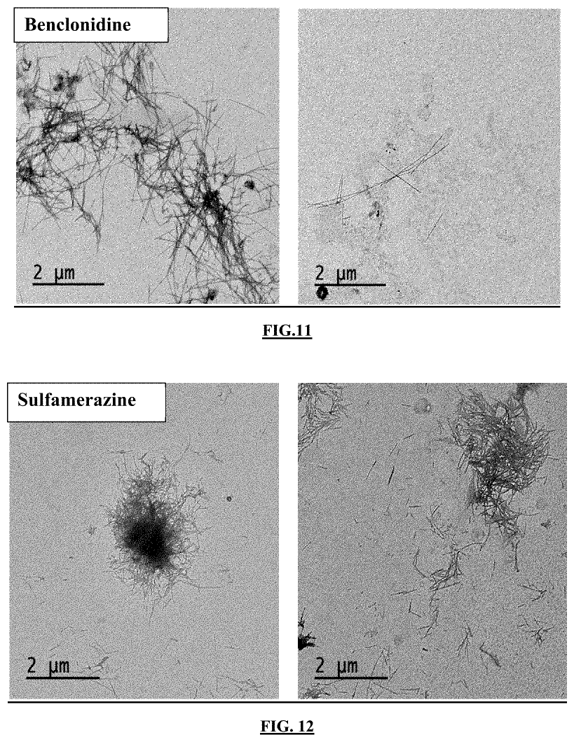

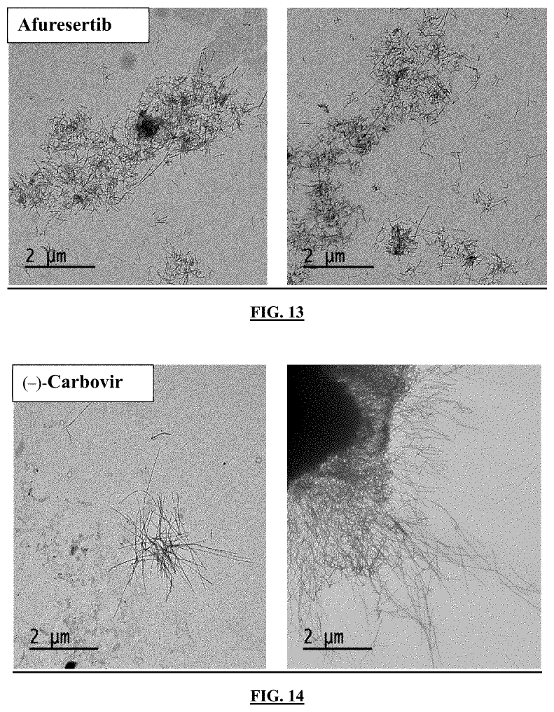

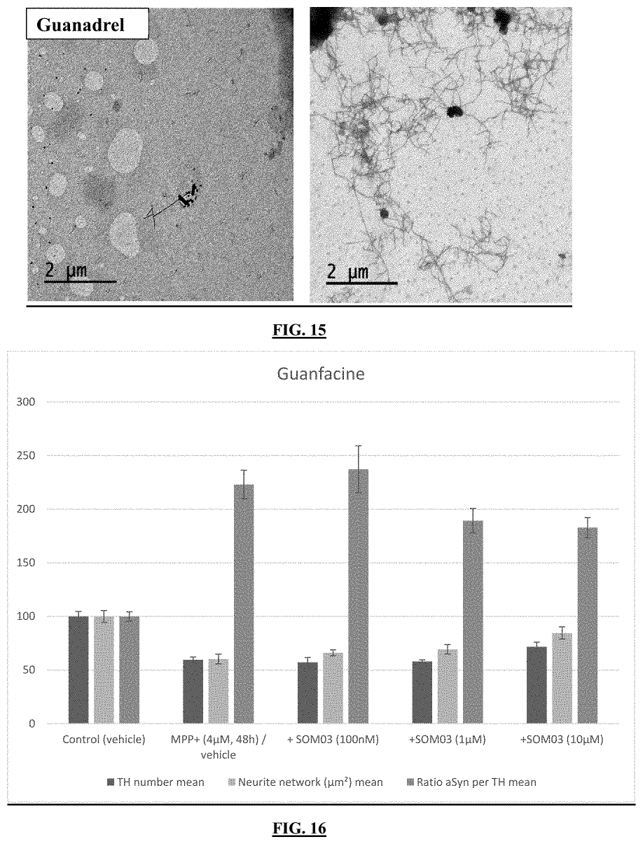

Finally, Transmission Electronic Microscopy (TEM) experiments confirmed the effects of the compounds on α-syn aggregation. TEM images of untreated samples showed a higher number of fibrils when compared with samples treated with the positive hits identified by Th-T fluorescence and Light Scattering measurements (FIG. 3-9 and FIG. 10-15).

### B. Efficacy in a Parkinson Disease Model

The efficacy of 5 compounds was assessed in a primary culture of dopaminergic neurons injured with a mitochondrial toxin (MPP+), which is often used to experimentally mimic the pathology of Parkinson disease (FIG. 16 to 20).

### Primary Culture of Mesencephalic Neurons

To obtain rat dopaminergic neurons, pregnant female rats (Wistar, Janvier Labs, France) of 15 days of gestation were killed using a deep anesthesia in a CO2 chamber followed by a cervical dislocation. Midbrains obtained from 15-day-old rat embryos were dissected under microscope and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, was used for the cell preparations.

The midbrains were dissociated by trypsinization for 20 min at 37° C. (solution at a final concentration of 0.05% trypsin and 0.02% EDTA). The reaction was stopped by adding Dulbecco's modified Eagle's medium (DMEM) containing DNAase I grade II (0.5 mg/mL) and 10% of fetal calf serum (FCS). Cells were then mechanically dissociated by 3 passages through a 10 ml pipette. Cells were then centrifuged at 180×g for 10 min at +4° C. on a layer of BSA (3.5%) in L15 medium. The supernatant was discarded and the cell pellet was re-suspended in a defined culture serum-free medium consisting of Neurobasal (Invitrogen) supplemented with B27 (2%), L-glutamine (2 mM) and 2% of PS solution and 10 ng/ml of Brain-derived neurotrophic factor (BDNF) and 1 ng/mL of Glial-Derived Neurotrophic Factor (GDNF). Viable cells were counted in a Neubauer cytometer using the trypan blue exclusion test. The cells were seeded at a density of 40.000 cells/well in 96 well-plates (pre-coated with poly-L-lysine) and maintained in a humidified incubator at 37° C. in 5% CO2/95% air atmosphere.

Half of the medium was changed every 2 days with fresh medium. On 96-wells plates, only 60 wells were used, the first columns and first lines were not used to avoid any edge effect, the empty wells were filled with sterile water.

### Test Compound Application and MPP+ Exposure

Test compounds were tested on culture in 96-well plates (n=6 culture wells per condition).

Vehicle: Culture medium (0.1% DMSO).

Pre-incubation: On day 6 of culture, the compounds (from 10 nM to 10 μM) were dissolved in DMSO and then in culture medium, and were pre-incubated with primary dopaminergic neurons for 1 hour or 4 hours before the application of MPP+.

Injury: One or four hours after the application of the test compounds, MPP+ was added to a final concentration of 4 μM, diluted in control medium still in presence of compounds for 48 hours.

### End-Point Evaluation

48 hours after the intoxication, the cell culture supernatant was removed, and the cells were fixed by a solution of 4% paraformaldehyde in PBS, pH=7.3 for 20 min at room temperature. The cells were washed twice in PBS, and then permeabilized. Non-specific sites were blocked with a solution of PBS containing 0.1% saponin and 1% FCS for 15 min at room temperature.

Immunostaining: TH and α-syn

The cultures were incubated with:
A monoclonal anti-Tyrosine Hydroxylase (TH) antibody produced in mouse at dilution of 1/10000 in PBS containing 1% FCS, 0.1% saponin, for 2 hours at room temperature.
a polyclonal anti-alpha synuclein (α-syn) antibody produced in rabbit at dilution of 1/200 in PBS containing 1% FCS, 0.1% saponin, for 2 hours at room temperature.

These antibodies were revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 hour at room temperature.

### Automatic Computer Analysis

For each condition, 20 (for the TH survival and α-syn readouts) pictures representing the whole well area, were automatically taken using ImageXpress® (Molecular Devices) at 10× magnification (20 pictures, for TH and α-syn into TH neurons) using the same acquisition parameters. From images, analyses were directly and automatically performed by Custom Module Editor® (Molecular Devices). The following read-outs were measured:
Analysis of total number of THneurons (TH positive neurons),
total length of neurite network of TH positive neurons (in μm),
α-syn aggregation (overlapping between TH and α-syn staining),

### Statistical Analysis

Results are expressed in percentage of control. All values show the mean+/−SEM (standard error of the mean) from 4-6 wells per condition.

### Results

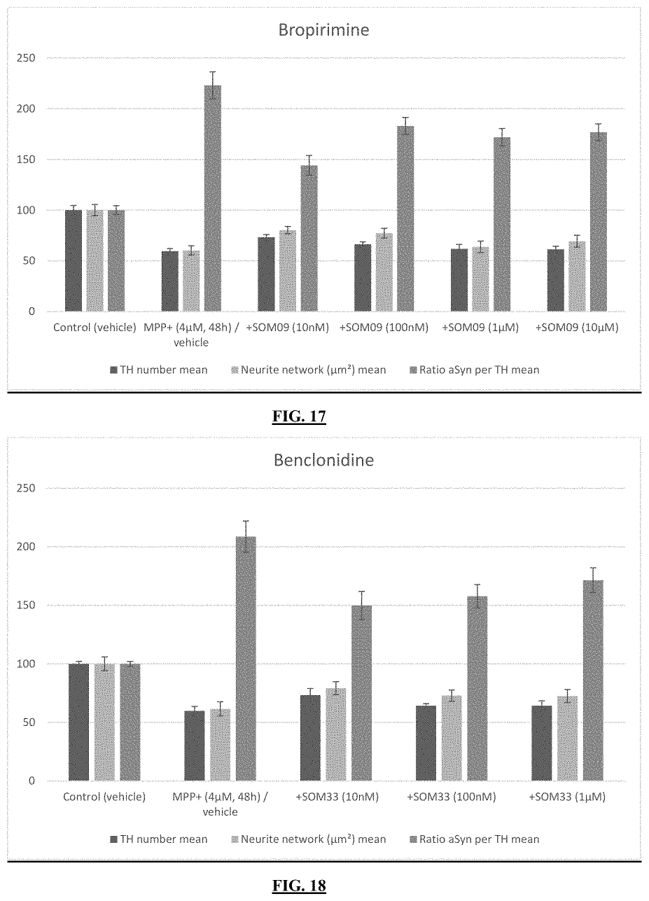

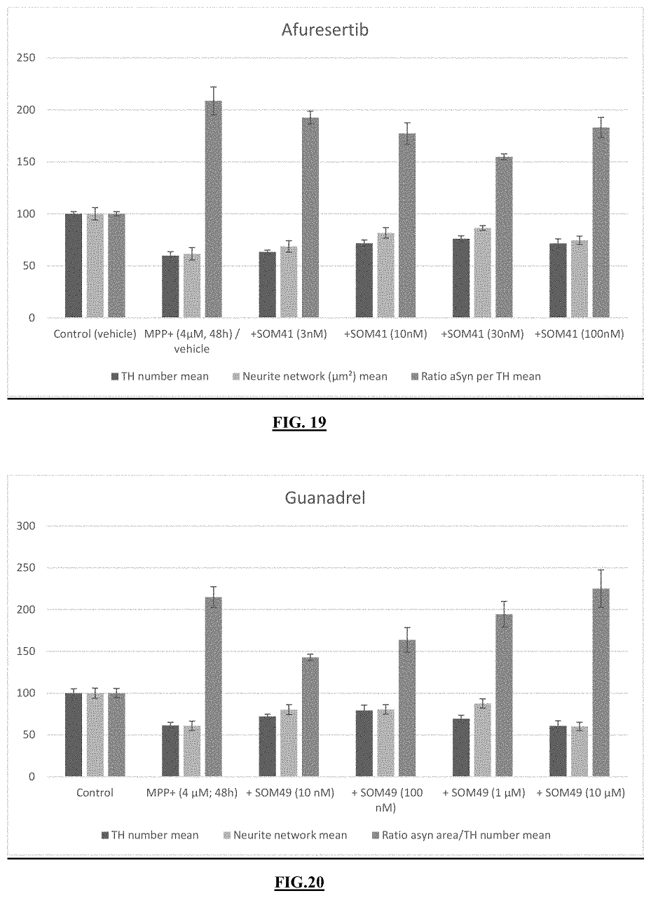

Treatment with MPP+ caused a decrease in the number of dopaminergic neurons (TH number) and a reduction of their neurite network, accompanied with an increase of α-syn aggregation (ratio a-syn area/TH number). Pre-treatment with guanfacine (FIG. 16), bropirimine (FIG. 17), benclonidine (FIG. 18), afuresertib (FIG. 19) or guanadrel (FIG. 20) at different concentrations partially prevented the loss of dopaminergic neurons and decreased α-syn aggregation triggered by the MPP+ insult, confirming that these compounds have a neuroprotective effect on dopaminergic neurons after an injury with MPP+.

1.-45. (canceled)

46. A method for the treatment and/or prevention of synucleinopathies wherein a compound selected from the group consisting of N-[1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one, N-(diaminomethylidene)-2-(2,6-dichlorophenyl)acetamide and 2-(1,4-dioxaspiro[4.5]decan-2-ylmethyl)guanidine, or a pharmaceutically acceptable salt thereof, is administered to a patient in need thereof.

47. The method according to claim 46, wherein the compound is N-[1-amino-3-(3-fluorophenyl)propan-2-yl]-5-chloro-4-(4-chloro-1-methyl-1H-pyrazol-5-yl)thiophene-2-carboxamide, or a pharmaceutically acceptable salt thereof.

48. The method according to claim 46, wherein the compound is [2-(2,6-dichloroanilino)-4,5-dihydroimidazol-1-yl]-phenylmethanone.

49. The method according to claim 46, wherein the compound is 2-amino-5-bromo-4-phenyl-1H-pyrimidin-6-one.

50. The method according to claim 46, wherein the compound is N-(diaminomethylidene)-2-(2,6-dichlorophenyl)acetamide, or a pharmaceutically acceptable salt thereof.

51. The method according to claim 46, wherein the compound is 2-(1,4-dioxaspiro[4.5]decan-2-ylmethyl)guanidine, or a pharmaceutically acceptable salt thereof.

52. The method according to claim 46, wherein the synucleinopathy to be treated and/or prevented is selected from the group consisting of Parkinson's disease (PD), Multiple System Atrophy (MSA), and Dementia with Lewy Bodies.

53. The method according to claim 52, wherein the synucleinopathy to be treated and/or prevented is Parkinson's Disease (PD).

54. A method for the treatment and/or prevention of synucleinopathies wherein a combination comprising two or more compounds selected from the group consisting of 1,2-bis[(4-chlorophenyl)methylideneamino]guanidine, N-[1-(1-naphthyl)ethyl]-3-[3-(trifluoromethyl)phenyl]propan-1-amine, N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, 4-[2-amino-6-(cyclopropylamino)-9H-purin-9-yl]-2-cyclopentene-1-methanol, 4-amino-N-(4-methylpyrimidin-2-yl)benzenesulfonamide, levodopa, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-03, PD-01, LuAF-82422, NPT-088, UB-312, and Anle138b or a pharmaceutically acceptable salt thereof, wherein at least one of the compounds in the combination is selected from the group consisting of N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, 2-amino-5-bromo-4-phenyl-1H-pyrimidin-6-one and 2-(1,4-dioxaspiro[4.5]decan-2-ylmethyl)guanidine, or a pharmaceutically acceptable salt thereof, is administered to a patient in need thereof.

55. The method according to claim 54, wherein the combination comprises at least one compound selected from N-[1-amino-3-(3-fluorophenyl)propan-2-yl]-5-chloro-4-(4-chloro-1-methyl-1H-pyrazol-5-yl)thiophene-2-carboxamide or a pharmaceutically acceptable salt thereof, N-[(2S)-1-amino-3-(3-fluorophenyl)propan-2-yl]-5-chloro-4-(4-chloro-1-methyl-1H-pyrazol-5-yl)thiophene-2-carboxamide or a pharmaceutically acceptable salt thereof, N-[1-amino-3-(3-fluorophenyl)propan-2-yl]-5-chloro-4-(4-chloro-1-methyl-1H-pyrazol-5-yl)thiophene-2-carboxamide hydrochloride, N-[(2S)-1-amino-3-(3-fluorophenyl)propan-2-yl]-5-chloro-4-(4-chloro-1-methyl-1H-pyrazol-5-yl)thiophene-2-carboxamide hydrochloride, [2-(2,6-dichloroanilino)-4,5-dihydroimidazol-1-yl]-phenylmethanone, 2-amino-5-bromo-4-phenyl-1H-pyrimidin-6-one, 2-(1,4-dioxaspiro[4.5]decan-2-ylmethyl)guanidine or a pharmaceutically acceptable salt thereof, 2-(1,4-dioxaspiro[4.5]decan-2-ylmethyl)guanidine hydroiodide, or 2-(1,4-dioxaspiro[4.5]decan-2-ylmethyl)guanidine sulfate.

56. A method for the treatment and/or prevention of synucleinopathies wherein a combination comprising two or more compounds selected from the group consisting of 1,2-bis[(4-chlorophenyl)methylideneamino]guanidine, N-[1-(1-naphthyl)ethyl]-3-[3-(trifluoromethyl)phenyl]propan-1-amine, N-[(2S)-1-amino-3-(3-fluorophenyl)propan-2-yl]-5-chloro-4-(4-chloro-1-methyl-1H-pyrazol-5-yl)thiophene-2-carboxamide, [2-(2,6-dichloroanilino)-4,5-dihydroimidazol-1-yl]-phenylmethanone, methyl N-(6-propoxy-1H-benzimidazol-2-yl) carbamate, 4-(2-(diaminomethylidene)hydrazinyl)phenyl]iminothiourea, N-(diaminomethylidene)-2-(2,6-dichlorophenyl)acetamide, 2-amino-5-bromo-4-phenyl-1H-pyrimidin-6-one, 2-(1,4-dioxaspiro[4.5]decan-2-ylmethyl)guanidine, 5-[2-[[3-(1,3-Benzodioxol-5-yl)-1-methylpropyl]amino]-1-hydroxyethyl]-2-hydroxybenzamide, 2-amino-9-[4-(hydroxymethyl)cyclopent-2-en-1-yl]-1H-purin-6-one, 4-[2-amino-6-(cyclopropylamino)-9H-purin-9-yl]-2-cyclopentene-1-methanol, 4-amino-N-(4-methylpyrimidin-2-yl)benzenesulfonamide, carbidopa, benserazide, entacapone, opicapone, amantadine, pimavanserin, rotigotine, ropinirole, apomorphine, rasagiline, safinamide, istradefylline, KW-6356, donepezil, rivastigmine, liraglutide, prasinezumab, venglustat, dipraglurant, mesdopetam (IRL-790), SEP-363856, cinpanemab, posiphen, MEDI-1341, UCB-0599, ABBV-0805, NPT-20011, PD-03, PD-01, LuAF-82422, NPT-088, UB-312, and Anle138b or a pharmaceutically acceptable salt thereof, wherein at least one of the compounds in the combination is N-(diaminomethylidene)-2-(2,6-dichlorophenyl)acetamide or a pharmaceutically acceptable salt thereof, is administered to a patient in need thereof.

57. The method according to claim 56, wherein the combination comprises N-(diaminomethylidene)-2-(2,6-dichlorophenyl)acetamide hydrochloride.

58. The method according to claim 54, wherein the synucleinopathy to be treated and/or prevented is selected from the group consisting of Parkinson's disease (PD), Multiple System Atrophy (MSA), and Dementia with Lewy Bodies.

59. The method according to claim 56, wherein the synucleinopathy to be treated and/or prevented is selected from the group consisting of Parkinson's disease (PD), Multiple System Atrophy (MSA), and Dementia with Lewy Bodies.

60. The method according to claim 46, wherein the compound is N-[(2S)-1-amino-3-(3-fluorophenyl)propan-2-yl]-5-chloro-4-(4-chloro-1-methyl-1H-pyrazol-5-yl)thiophene-2-carboxamide, or a pharmaceutically acceptable salt thereof.

61. The method according to claim 46, wherein the compound is N-[1-amino-3-(3-fluorophenyl)propan-2-yl]-5-chloro-4-(4-chloro-1-methyl-1H-pyrazol-5-yl)thiophene-2-carboxamide hydrochloride.

62. The method according to claim 46, wherein the compound is N-[(2S)-1-amino-3-(3-fluorophenyl)propan-2-yl]-5-chloro-4-(4-chloro-1-methyl-1H-pyrazol-5-yl)thiophene-2-carboxamide hydrochloride.

63. The method according to claim 46, wherein the compound is N-(diaminomethylidene)-2-(2,6-dichlorophenyl)acetamide hydrochloride.

64. The method according to claim 46, wherein the compound is 2-(1,4-dioxaspiro[4.5]decan-2-ylmethyl)guanidine hydroiodide.

65. The method according to claim 46, wherein the compound is 2-(1,4-dioxaspiro[4.5]decan-2-ylmethyl)guanidine sulfate.
